(19) United States
(12) Patent Application Publication
(10) Pub. No.: US 2020/0397065 A1
(43) Pub. Date: Dec. 24, 2020

(54) COMFORT ARTICLE TO PREVENT FEET FROM SLIDING IN SHOES

(71) Applicant: Lucille Jackson, Upper Saddle River, NJ (US)

(72) Inventor: Lucille Jackson, Upper Saddle River, NJ (US)

(21) Appl. No.: 16/444,069

(22) Filed: Jun. 18, 2019

(51) Int. Cl. A41B 11/00; A61K 9/70; A61K 31/125; A61K 31/045; A61K 31/618; A61K 31/165; A61K 31/167; A61K 31/196

(52) U.S. Cl. CPC A41B 11/008; A61K 9/7038; A61K 31/125; A61K 31/196; A61K 31/618; A61K 31/165; A61K 31/167; A61K 31/045

(57) ABSTRACT

A comfort article may comprise a proximal layer that adheres to the skin of a foot, and a non-slip distal layer with a high coefficient of friction that prevents footwear from sliding with respect to the foot without adhering to or sticking to the shoe. The proximal layer comprises a sticky adhesive surface or dry adhesive. The comfort article is cutout into various shapes and applied to one or more locations on a user's foot at a point of contact within the shoe that is intended to prevent the foot from moving with respect to the shoe. The comfort articles may also be supplied as a kit with various precut devices configured to fit certain types of footwear and accommodate user preferences. A cushion layer(s) may be interposed between the proximal layer and distal layer.

### FIELD OF THE DISCLOSURE

This disclosure relates to articles for feet and more particularly to a comfort article configured to minimize sliding or slippage between a foot and footwear.

### BACKGROUND

Footwear includes practical and functional designs as well as high fashion and ornamental style. Certain shoe designs may focus on aesthetics and may sacrifice certain function, such as long term comfort to the wearer. Footwear may not merely be a functional item, they can be a fashion statement or an accessory. For example, certain shoes do not hold the feet firmly in place and allow the foot to move relative to the shoe thereby causing rubbing or chafing and creating blisters. Shoes may allow the foot to slide forward in an uncomfortable position when the user is standing or walking for extended periods of time.

Securely affixing a shoe to a wearer's foot can be a significant problem in footwear. Several products have been developed in an attempt to address this issue. For example, US20180042342A1 to Kleinsasser disclose a tape product with adhesive on both surfaces that adheres to the shoe and the skin of the person wearing the shoe. But, there are several shortcomings to these double sided-adhesive devices. For example, when the foot or device is removed from the shoe, some adhesive residue may remain in the shoe leaving an unsightly spot in the shoe. Another issue with double sided tape products is that when the foot is removed from the shoe, either an adhesive residue will remain on the foot or the tape itself will remain on the foot, and in either case, an exposed adhesive surface on the users foot will tend to pick up dust, hair, carpet fibers, and the like on the floor which must be removed. In the event the user wants to reinsert the foot into the shoe, the detritus may spoil the adhesive tack and cause discomfort if solid material is picked up from the floor. Also, simply walking with an adhesive surface on the bottom of the foot is likely to be uncomfortable and possibly even dangerous as a tripping hazard.

Another product offered in an attempt to address the issue of foot slippage in shoes is “Non-Slip Grip Pads for High Heel Shoes, Boots and Sandals,” which consists of a pad that adheres to a shoe and has a non-slip surface in contact with the foot. Over time, these solutions become ineffective and unsightly when shoes are removed. The pads can flatten, fall out, or begin to smell from foot perspiration. In this type of device, when the pad is removed from the shoe, there is residue from the pad itself or the adhesive and the shoe is uncomfortable to wear from the tackiness. The shoe can also become unsightly because debris such as dust, dirt, and hair can stick to the adhesive residue inside the shoe. This can also make the shoe uncomfortable to step in. Many of these kinds of devices may have sufficient bulk to change the fit of the shoe, which is also undesirable and can lead to discomfort.

Furthermore, some devices that stick to the interior of a shoe have minimal adhesive tack and can fall out or shift during use. A user may not be able to restore the pad in place and must hobble around with an ill-fitting shoe.

In addition, more recently, the popularity of rental apparel is increasing. Therefore, the existing art of products that adhere to a shoe are undesirable.

Various products that adhere to the skin of the foot have been disclosed and sold. A popular skin adherence product for feet is generically called “moleskin” and is designed to prevent and treat foot blisters by shielding and protecting skin from a contact point in a shoe that causes a friction injury. See for example Nakagawa et al. in US20150265742.

Other skin adhesion products have been disclosed for bandages, in for example U.S. Pat. No. 4,302,500 and for kinesiology tape in US20120232448. A golf gripping bandage is disclosed in U.S. Pat. No. 6,049,022.

Other devices to prevent slippage of feet in shoes include US20130047464 which discloses a high friction device for high-heel shoes with rings that toes are inserted into. Various devices are designed to adhere to the shoe and have a high friction surface or cushion intended to reduce foot slippage.

However, improvements are needed.

### SUMMARY OF THE DISCLOSURE

The present disclosure relates to a comfort article for the foot that is thin, disposable, discreet, and does not stick to the inside of a shoe causing potential damage to the shoe. At the same time, the comfort article of the present disclosure does not make a shoe more uncomfortable and does not employ any unsightly accessories that harm the aesthetics of footwear.

A comfort article may comprise a proximal layer that adheres to a surface of a foot and a distal layer that prevents footwear from sliding with respect to the foot without adhering to the shoe. By the phrase “without adhering to the shoe,” it is meant that there is no glue or adhesive material on the comfort article that sticks to a surface of a shoe (e.g., there is no material transference that takes place between the comfort article and the shoe. Additionally or alternatively, the proximal surface may be adhered to a sock, pantyhose, or other garment worn in close contact with the skin of the foot. The tack or level of adhesive ability may be adjusted for a particular application. For example, levels of adhesive strength may be configured for skin, sock, or other materials.

A comfort article may be configured to minimize sliding or slippage of a foot within a shoe. The comfort article may comprise proximal layer and distal layer, wherein the proximal layer comprises an adhesive surface that adheres to the skin of the foot, and wherein the adhesive surface is applied to the skin of a foot at a point of contact within a shoe, and wherein the adhesive surface adheres to the foot. A distal layer of the comfort article may comprise a non-adhesive, friction surface configured to minimize sliding or slippage of a foot within a shoe, without adhering to the shoe. Alternatively, the proximal layer of the comfort article is a gripping surface that securely grips/contacts the skin of the foot without sticking to the skin.

The proximal and distal layers may further comprise a plurality of surfaces/layers bonded to one another.

A comfort article may comprise a plurality of layers having a proximal layer, a distal layer, and a cushion layer interposed in between the proximal surface and distal surface. The proximal layer may comprise an adhesive surface and a substrate or fabric. The distal layer may comprise a gripping surface/material and a substrate or fabric.

A method for preventing a foot from sliding or slipping in a shoe may comprise the use of a comfort article having a proximal layer and a distal layer, wherein the proximal layer comprises an adhesive surface that adheres the comfort article the skin or a gripping surface that securely grips/makes contact with a surface of the foot without sticking to the skin or material transference. A distal layer of the comfort article may comprises a non-adhesive, friction surface configured to prevent the foot from moving with respect to the shoe, without adhering to the shoe or material transference between the article and the shoe.

In an alternative to the apparatus and methods described above, the proximal layer of the comfort article adheres to a sock or pantyhose, or other garment worn in close contact with the skin of the foot.

### DESCRIPTION OF THE DRAWINGS

FIGS. 1A, 1B, and 1C are bottom views of feet with various aspects of the comfort articles.

FIG. 2 is a view of the back of a foot with an aspect of the comfort article.

FIGS. 3A, 3B, and 3C are cross section views of various aspects of the comfort articles.

FIG. 4 is a side perspective view of an aspect of the comfort article showing the gripping and adhesive sides.

FIG. 5 is a perspective view showing two feet with different aspects of comfort articles.

### DETAILED DESCRIPTION

A comfort article may be configured to minimize sliding or slippage of a foot within a shoe. The comfort article may comprise proximal layer and distal layer, wherein the proximal layer comprises an adhesive surface that adheres to the skin of the foot, and wherein the adhesive surface is applied to the skin of a foot at a point of contact within a shoe, and wherein the adhesive surface adheres to the foot. A distal layer of the comfort article may comprise a non-adhesive, friction surface configured to minimize sliding or slippage of a foot within a shoe, without adhering to the shoe. Alternatively, the proximal layer of the comfort article is a gripping surface that securely grips/contacts the skin of the foot without sticking to the skin.

As defined in this disclosure, a “comfort article” may be or comprise a planar flexible material, such as a sheet, with one or more layers of planar materials. As an example, two or more layers may be bonded together. The “proximal” layer may be in contact with the skin of a person (or garment disposed adjacent the skin). A “distal” layer may be defined as the opposite/outer layer of the comfort article that is in contact with the footwear. The proximal and distal layers may comprise pluralities of bonded layers. The article may be precut into various shapes, such as rectangles, ovals, circles, or other geometric shapes, of various sizes to fit onto a foot. The term “slippage” means that a person's foot (also referred to herein as a user's foot) moves with respect to the shoe. Slippage in a shoe can cause discomfort, a sense of instability for the wearer, and friction injuries, such as chafing or blisters. “Footwear” and “shoe” are used synonymously herein.

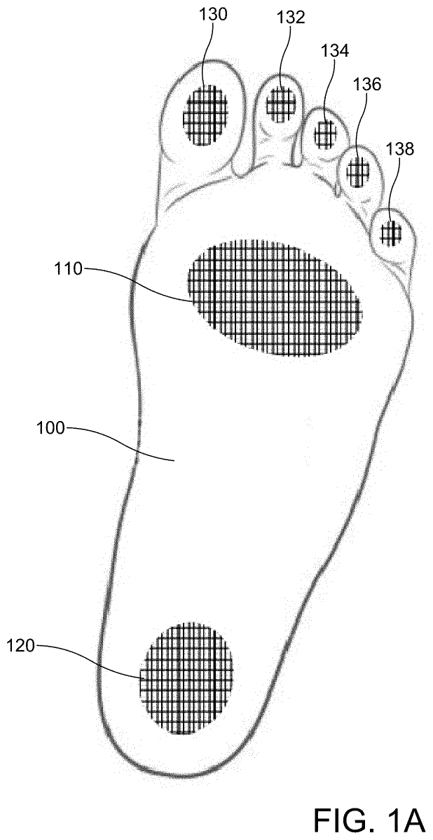

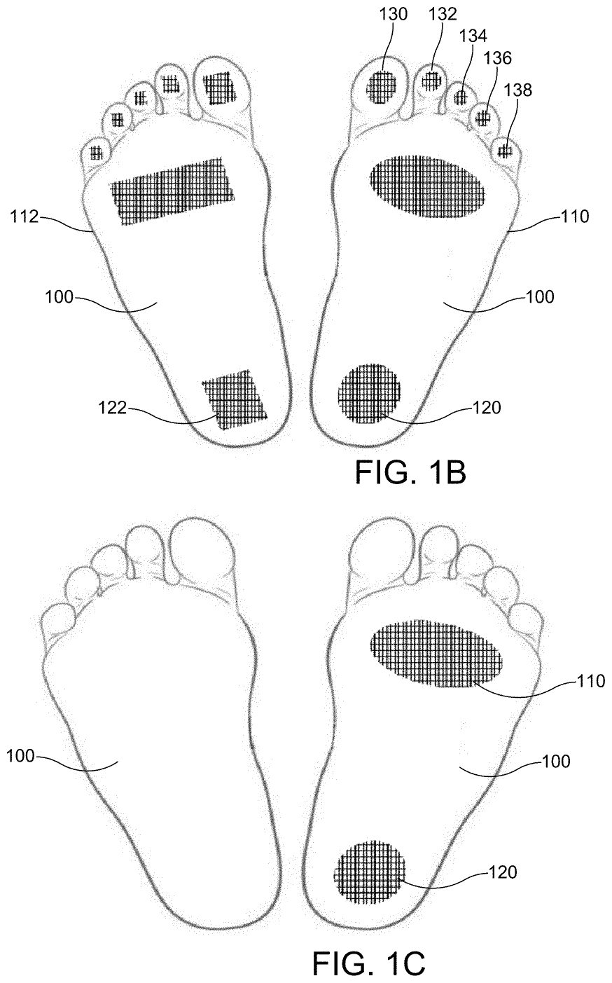

The comfort article of the present disclosure may be applied/affixed to the heel, the ball of the foot or any location on the forefoot, the toes, or to the back of the foot. The comfort article may be applied to any portion of the foot or body where contact with a surface (e.g., of the footwear) is expected. Example aspects are shown in FIGS. 1A-C, and 2.

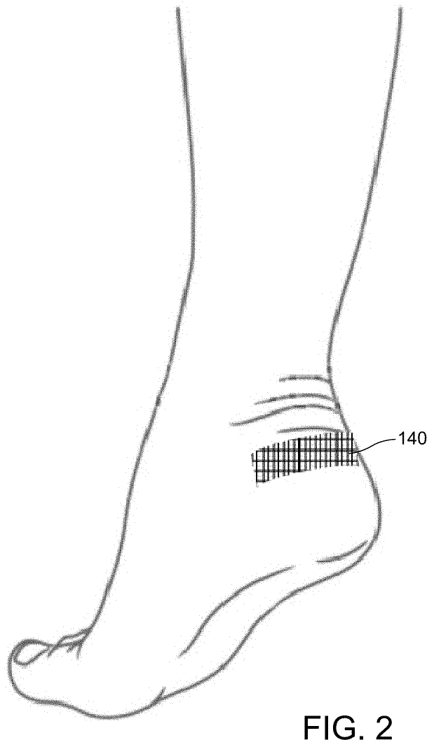

FIGS. 1A-1C show a comfort article applied to various locations on the bottom of a foot 100. The comfort article may be applied to the ball 110 of the foot 100 or to the heel region 120 on the bottom of the foot 100. The comfort article may be applied to the toes 130-138. FIG. 2 shows a comfort article affixed to the rear heel 140 of a foot. The comfort article illustrated in each of FIGS. 1A-1C and/or FIG. 2 may be provided as a kit.

A wearer may choose not to use the articles in all of these locations. For example, a user may find that the comfort article applied to only one location is sufficient, such as the ball of the foot. The comfort articles may be applied to one or more locations on a user's feet. In addition to the locations illustrated in FIGS. 1A-C, and 2, the comfort articles may be applied to other locations also, such as the outer arch of the foot, or an upper surface of the foot.

The comfort article may be cutout or precut as an oval 110 and 120 or circular, as shown in 130, 132, 134, 136, and 138 (as shown in FIG. 1A and the illustration on the right in FIG. 1B). The comfort article may be cutout or precut as a rectangle 111, or square 121, 131, 133, 135, 137 and 139 (as shown on the left in FIG. 1B). Other cutout shapes are of course possible, for example rectangles or squares or irregular shapes that may be engineered to adhere/affix to various locations and foot sizes, or that may be designed with a fashion purpose, such as a fish, flower, or butterfly shape. Different shapes may be made for different areas of the foot. For example, an article for the ball of the foot will be wider, a device for the bottom of the heel will be narrower, an article for the back of the heel will be longer and thinner, and an article for the toes will be smaller.

The comfort articles may be provided as a kit configured to suit a particular type of footwear. For example, precut articles as embodied in FIG. 1A, along with precut articles for the left foot (not shown in FIG. 1A) may be included in a kit intended specifically for high-heeled sandals.

A kit intended for use with a boot or sneaker may comprise comfort articles for the upper surface and outer arches of the foot. A kit intended for use with a covered pump may comprise comfort articles for each toe, the balls of each foot, the bottom heel and the rear of each foot. Further, kits may be supplied with comfort articles intended to fit specific shoe sizes or ranges of shoe sizes. Kits may be supplied with vials of oil or a like substance to make it easier to remove sticky adhesive or residue from the user's skin.

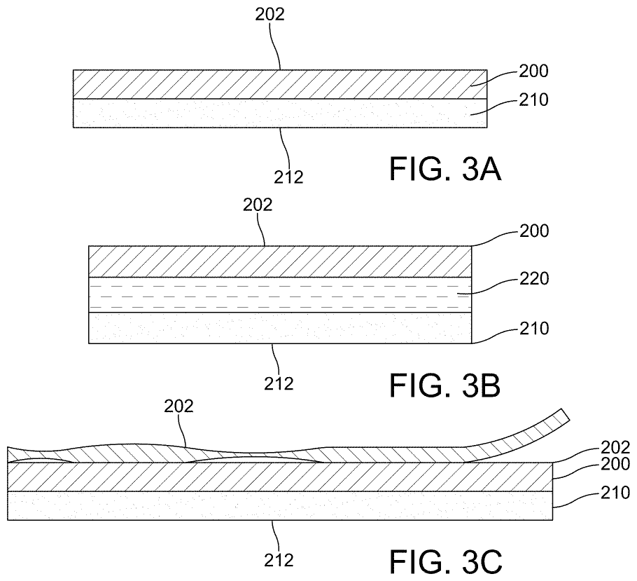

Example comfort articles are shown schematically in FIGS. 3A, 3B and 3C.

In FIG. 3A, an example two-ply article is depicted as a cross section. A proximal layer 200 may comprise an adhesive or gripping surface 202 that adheres the comfort article to or securely grips the foot of a person wearing a shoe. A distal layer 210 may comprise a non-adhesive, friction surface 212 that resists relative movement of a surface in contact with friction surface 212.

FIG. 3B shows an example three-layer structure comprising a proximal layer 200, a distal layer 210 comprising a high friction surface, and a cushion layer 220 interposed between the proximal layer 200 and the distal layer 210. Additional layers may be interposed between the proximal layer 200 and the distal layer 210.

FIG. 3C shows an example two-layer article similar to the article depicted in FIG. 3A, where the proximal layer comprises an adhesive surface that sticks to the wearer's skin. As shown in FIG. 3C, the article may comprise an adhesive backing 204. The backing 204 may be removed prior to application of the comfort article to the foot. Removing the backing exposes the adhesive surface 202. In FIG. 3C, backing 204 is depicted as partially removed. The adhesive backing 204 may be employed with the three-layer article shown in FIG. 3B.

Where an adhesive surface and adhesive backing 204 are employed, the backing may include a tab or an extension to make removal easier. Many backing materials are firmly attached to the underlying material, e.g., proximal layer 200, and removal of the backing can be difficult. The use of fingernails or a tool may be required. Many people, for example, may have expensive fingernail treatments that they don't want damaged by this kind of use. Thus, the inclusion of a tab on the backing to remove it easily and without tools may be desirable. Where a dry/microfiber adhesive is employed, the backing may not be required.

An example of an adhesive material for the proximal layer 200 may be or comprise kinesiology tape, available from many sources. The adhesive material 200 may be medical grade and/or hypoallergenic. Other materials with an adhesive coating, such as a bandage-type of material, may be similarly suitable for the comfort articles. Additionally or alternatively, other adhesive materials may be used. The adhesive may be adjusted for different conditions such as the humidity or the amount of moisture on a person's foot. A person that sweats profusely, for example, may require a stronger adhesive or a more waterproof adhesive.

Alternatively, the proximal layer 200 comprises a gripping surface/material that securely grips the foot of the wearer without sticking to it or material transference. The proximal layer 200 may comprise a dry, reusable material that is capable of securing skin to footwear without leaving residue behind. The proximal layer may comprise a microfiber adhesive made from polypropylene or keratin material. The microfiber adhesive may comprise an array of rigid microscopic polypropylene fibers that serve as contact points capable of adhering to a surface without material transference and can be removed without leaving a residue behind. The microfibers of the dry adhesive have anti-self-mating properties, which means they do not stick to/adhere to one another. The microfibers may be between 15-20 micrometers long with a diameter of approximately 0.6 micrometers. Microfiber adhesives employs directional attachment to secure one surface to another i.e. the gripping force is in a direction that is generally parallel to another surface.

In order to remove the dry adhesive from the shoe, the user peels it off/applies force in the perpendicular direction. The microfiber adhesive has a high sheer strength and therefore does not slide easily when affixed to a parallel surface. The microfiber adhesive has a low peel strength which is the measure of force required to part two bonded materials. For example, the microfiber adhesive may have a shear strength between 10-40 N/cm² and a peel strength between 0.1-2 N/cm². The shear and peel strength properties allow the material to very securely grip a surface it is parallel to but be easily removed/peeled off by applying a perpendicular force. Properties of an exemplary microfiber adhesive include a high pull-off force to preload ratio, anti-self-mating properties and low detachment force.

The proximal layer 200 may be in direct contact with the wearer's skin. In an alternative aspect, the proximal layer 200 is not in direct contact with the skin, but contacts a sock, pantyhose, or other garment worn in close contact with the skin. In this disclosure, when reference is made to the proximal layer contacting skin of the foot, the alternative contact with an intermediary garment such as a sock, pantyhose, or other garment worn in close contact with the skin is also intended. The tack or level of adhesive ability may be adjusted for a particular application. For example, levels of adhesive strength may be configured for skin, sock, or other materials. Alternatively or additionally, characteristics of the adhesive may be based on the target surface for adhesion. For example, if the target surface is skin, the adhesive (and/or the comfort article) may be hypoallergenic. As a further example, if the target surface for adhesion is a sock or garment, hypoallergenic characteristic may not be necessary.

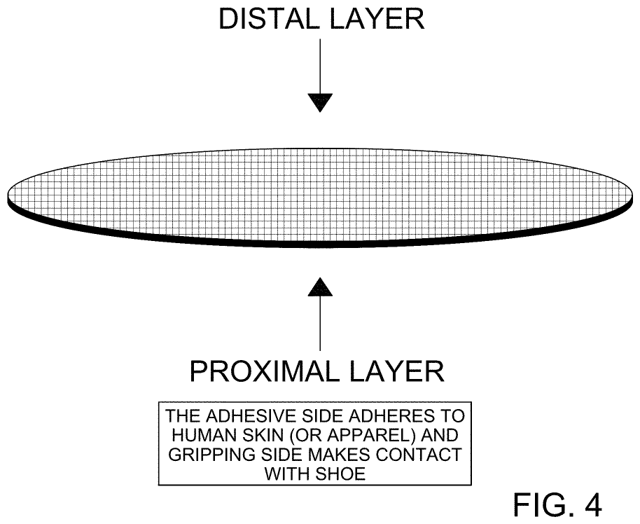

FIG. 4 illustrates an example precut comfort article with a circular shape. The distal layer 210 is opposite the proximal layer 200.

The distal layer 210 may comprise a non-slip gripping surface/material that is firm, stiff, and durable. The distal layer 210 may comprise a thermoplastic polyurethane polymer that is abrasion resistant meaning it is able to resist surface wear caused by rubbing/contact with another material. The distal layer 210 may comprise a similar or the same microfiber adhesive/gripping material to the proximal layer described above. The distal layer 210 may comprise a thin polyester mesh fabric with suitable properties for gripping a surface of the shoe without adherence, sticking and material transference that can be removed without leaving a residue behind.

An example of a gripping material for the distal layer 210 is “3M™ TB731 gripping material,” which is a thermoplastic polyurethane gripping polymer with high abrasion resistance and a high coefficient of friction. The gripping material may have a coefficient of friction between 0.98 and 1.1 depending on the properties of the surface it is contacting. Other flexible gripping materials are commercially available and suitable for use if sufficiently flexible.

A cushion layer 220 may be a fabric, foam, gel, silicone or a shock absorbing material. The cushion layer should be thin enough not to distort the sizing and fit of the shoe. The cushion layer 220 may be made from and/or include a material or substance that provides additional benefits such as a pain reliever, odor-control, or a scent. Other layers, with or without the cushion layer 220, may be configured for odor control or seat management.

Regardless of the configuration of the layers of the comfort article of the present disclosure, it may be a thin product that does not distort the sizing of the shoe.

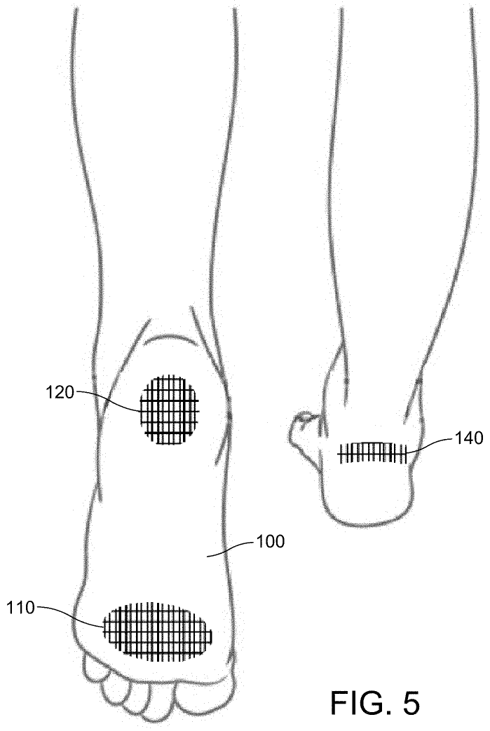

The comfort article may be supplied as a roll that the user can cut as desired. FIG. 5 is a perspective view showing the bottom of a left foot and the back of a right foot with different sizes and shapes of comfort articles. The comfort articles can be selected from precut shapes or cut out by the user from a roll of tape.

Generally, where the proximal layer 200 comprises an adhesive surface, the comfort article may be applied to clean and dry skin. A user may wish to cleanse the area where the adhesive surface is to be applied with rubbing alcohol. The comfort article may be supplied with an alcohol wipe, for example, a kit with comfort articles and an alcohol wipe. The kit for a specific type of footwear such as the high-heeled sandal described above may include an alcohol wipe. A kit may be supplied with options for the strength/stickiness of the proximal layer to accommodate user preferences. For example, regular hold adhesive, an extra strong hold adhesive, and dry adhesive versions of the comfort articles may be contained in various kits. A roll of the comfort article may also be supplied with these options.

As compared to other devices that adhere to the foot, for example moleskin, the comfort article has a high friction distal surface. Moleskin is intended generally to prevent and treat friction injuries, but only by protecting the skin from rubbing against a surface of the shoe. Moleskin does not have a gripping surface that is intended to stop movement of a foot relative to a shoe.

One advantage to the comfort article, compared to most prior art devices that address the same problem, is that this device does not harm the shoe or leave a potentially unsightly or damaging fixture attached to the shoe. The comfort article does not harm the shoe or involve any kind of adhesive surface that adheres to the shoe itself. In many applications, this can be an important benefit. For example, prior art devices with an adhesive may damage the shoe when removed. In another example, rented or borrowed footwear will not be harmed with the comfort article because nothing adheres to the shoe itself.

A further advantage to not using an article that adheres to the shoe itself is that conventional foot pads and insoles do not always stay positioned within a shoe and can become bunched and wrinkled, particularly after multiple uses. Or if not positioned correctly, the sticky effectiveness is not as good if not reapplied (see e.g., U.S. Pat. No. 5,737,776, 1:32-40).

The comfort article has other advantages. For example, compared to prior art devices that address the same problem, the comfort article is disposable and sanitary. The comfort article of the present disclosure is easily applied and removable. The dry adhesive may be reusable and movable. After use, the comfort article can be easily removed from the foot. This is also a factor in sanitation. The comfort articles need not be cleaned after use. Rather, they are simply removed and discarded. The comfort articles may be removed and reused. Prior art devices that adhere to/stick to the shoe may require cleaning.

The comfort article may include an odor control additive or a perfume to prevent unpleasant foot odors. For example, an antiperspirant may be added to the proximal layer. A perfume may be added to any layer of the comfort article.

The proximal layer may be medicated with a topical pain reliever such as a camphor, menthol, methyl salicylate, capsaicin, or lidocaine, or a combination of any of these ingredients. The proximal layer may include other medical substances such as diclofenac, other non-steroidal anti-inflammatories (NSAID's), or anti-inflammatory steroidal drugs. Holistic or natural treatments may be used, such as hemp-based products, cannabis-based products, CBD oils or the like.

Another advantage is that the comfort articles provide gripping traction that is invisible to the public. This is an aesthetic advantage. Because the comfort article is applied to the foot and not the shoe, it provides flexibility and discretion. No unusual accessories will normally be visible. In many instances, the comfort articles are completely hidden from view, for example, in placement on the bottom of the foot.

In accordance with an aspect of the present disclosure, by minimizing or preventing foot slippage, most people will feel more comfortable wearing shoes where slippage is a problem. At the same time, there is no increase in discomfort because the comfort article is sufficiently thin that it does not affect the size of a shoe or cause any kind of constriction or bulky insert that could be uncomfortable.

In accordance with an aspect of the present disclosure, the comfort article can prevent spreading of the toes which is a particular problem in high heel open toes shoes but can also be a problem in open toe shoes that are not high heeled. This is caused by the foot sliding forward in the shoe. Many users find that spreading of toes is uncomfortable, unsightly, or both. The comfort article, by preventing the foot from sliding or slipping in the shoe, can prevent this problem.

In accordance with an aspect of the present disclosure, the comfort articles may prevent the toes from being constricted in a closed toe shoe, in particular high heels, where the foot can slide forward. In accordance with an aspect of the present disclosure, by preventing the foot from sliding forward within the shoe, this problem may be addressed.

In accordance with an aspect of the present disclosure, the feet can be removed from a shoe and the person can walk relatively normally, without having, for example, an adhesive surface that sticks to floors, scratches floors, or attracts floor detritus that sticks to the foot after the foot from walking even short distances without the shoe. The materials used in the present disclosure will not affect gait and will not adhere to carpets and will not scratch floors.

Further, the comfort article may be available with distal layers of various colors. Colors may be made to match skin tones that a user can select. This can help by making the inventive comfort article less visible and more discreet. The comfort article may have a color contrasting to skin and may include, for example, a print or pattern, and in effect be a wearable fashion accessory.

This device may be used with any type of footwear, including fashion footwear such as high heels or sandals, or conventional shoes such as boots and sneakers that don't fit well. The term “conventional shoes” here means footwear with a full upper that encases the foot. This device may thus address the problem of footwear that feels satisfactory when first tried on, but after being worn for a number of hours begins to cause pain. If the pain is due to rubbing of the foot against a surface of the shoe, the comfort article will be of value.

1. A kit comprising:
a first comfort article configured in one or more of shape or size for a first portion of a foot of a wearer; and
a second comfort article configured in one or more of shape or size for a second portion of the foot of the wearer, wherein the second portion is spaced from the first portion,
wherein each of the first comfort article and the second comfort article comprises:
a proximal layer having an adhesive surface configured for adhesive contact with a surface of the foot; and
a distal layer disposed opposite the proximal layer, wherein the distal layer is configured for non-adhesive, removable contact with a surface of footwear worn on the foot, and wherein the distal layer comprises a non-adhesive, friction surface configured to minimize moving between the distal layer and the surface of the footwear without adhering to the footwear and without transferring residue to the footwear when the foot is removed from the footwear.

2. The kit of claim 1, further comprising an alcohol wipe for cleaning the surface of the foot where the adhesive surface is to be applied.

3. The kit of claim 1, wherein the first comfort article is configured in one or more of shape or size for a bottom heel portion or a rear heel portion of the foot.

4. The kit of claim 1, wherein the second comfort article is configured in one or more of shape or size for a forefoot portion or toes of the foot.

5. (canceled)

6. (canceled)

7. The kit of claim 1, wherein the proximal layer comprises a dry microfiber gripping material configured to grip the surface of the foot.

8. The kit of claim 1, wherein the proximal layer and the distal layer comprise one or more dry microfiber gripping materials.

9. The kit of claim 1, wherein the distal layer comprises a dry microfiber gripping material.

10. The kit of claim 1, wherein the distal layer comprises a thermoplastic polyurethane gripping polymer having a coefficient of friction between 0.98 and 1.1.

11. The kit of claim 1, wherein the distal layer comprises a thermoplastic polyurethane gripping polymer.

12. The kit of claim 1, wherein the proximal layer comprises kinesiology tape.

13. The kit of claim 1, further comprising at least one cushion layer interposed between the proximal layer and the distal layer.

14. The kit of claim 1, wherein one or more of the first comfort article or the second comfort article has a precut shape selected from an oval, circle, rectangle, square, or an irregular shape.

15. The kit of claim 1, further comprising an odor control additive or perfume disposed on one or more of the proximal layer or the distal layer.

16. The kit of claim 1, wherein the proximal layer comprises a topical pain reliever.

17. The kit of claim 16, wherein the topical pain reliever comprises one or more of camphor, menthol, methyl salicylate, capsaicin, or lidocaine, diclofenac, other non-steroidal anti-inflammatories (NSAID's), or anti-inflammatory steroidal drugs, or a combination of any of these ingredients.

18. The kit of claim 1, wherein the distal layer is provided in a color intended to match the skin tone of the user, or in another color or a print or pattern.

19. (canceled)

20. (canceled)

21. A kit comprising:
a first comfort article configured in one or more of shape or size for a first portion of a foot of a wearer; and
a second comfort article configured in one or more of shape or size for a second portion of the foot of the wearer, wherein the second portion is spaced from the first portion,
wherein each of the first comfort article and the second comfort article comprises:
a first layer having an adhesive surface configured for adhesive contact with one or more of a surface of the foot or an intermediary garment worn in close contact with a surface of the foot; and
a second layer disposed opposite the first layer, wherein the second layer is configured for non-adhesive, removable contact with a surface of footwear worn on the foot, and wherein the second layer comprises a non-adhesive, friction surface configured to minimize moving between the second layer and the surface of the footwear without adhering to the footwear or transferring residue to the footwear when the foot is removed from the footwear.

22. A kit comprising:
a plurality of comfort articles each of which is configured in one or more of shape or size for a portion of a foot of a wearer;
wherein each of the comfort articles comprises:
a first layer having an adhesive surface configured for adhesive contact with a surface of the foot; and
a second layer disposed opposite the first layer, wherein the second layer is configured for non-adhesive, removable contact with a surface of footwear worn on the foot, and wherein the second layer comprises a non-adhesive, friction surface configured to minimize moving between the second layer and the surface of the footwear without adhering to the footwear or transferring residue to the footwear when the foot is removed from the footwear.
